(19) United States
(12) Patent Application Publication
(10) Pub. No.: US 2019/0082945 A1
(43) Pub. Date: Mar. 21, 2019

(54) MEDICAL DEVICE WARMER WITH REMOVABLE INSERT

(71) Applicant: ADVENTIST HEALTH SYSTEM/SUNBELT, INC., Altamonte Springs, FL (US)

(72) Inventor: Steve Eubanks, Winter Park, FL (US)

(21) Appl. No.: 16/130,513

(22) Filed: Sep. 13, 2018

Related U.S. Application Data
(60) Provisional application No. 62/559,316, filed on Sep. 15, 2017.

(51) Int. Cl. A61B 1/12; A61B 1/00; G02B 23/24

(52) U.S. Cl. CPC A61B 1/127; G02B 27/0006; G02B 23/2476; A61B 1/00135

(57) ABSTRACT

A disposable insert for warming a medical device includes: a base having a canister compartment formed therein; a canister held within the canister compartment and having a chamber formed therein which defines a chamber opening; a resistive heater thermally bridged with the canister; a controller electrically coupled to the resistive heater and configured to provide electric current to the resistive heater; and an insert mating feature connected to the base and configured to releasably engage a corresponding mating feature of a housing, the insert mating feature including at least one beveled surface. A warmer for a medical device includes the insert with the insert mating feature releasable engaging a housing mating feature of a housing.

The present disclosure claims priority under 35 USC § 119 to U.S. Provisional Application 62/559,316, which was filed on Sep. 15, 2017 and is herein incorporated by reference.

The present invention relates to warmers and, in some particular embodiments, to warmers for medical devices such as endoscopes. The warmers can prevent fogging of endoscopes or similar instruments when inserted in a body.

Various types of medical or surgical scopes are used in procedures to allow a physician user to view the interior of a patient's body before, during, or after a surgical procedure. Some exemplary medical scopes include laparoscopes, thoracoscopes, endoscopes, or similar devices. Typical scopes can include a visualizing element, such as a camera, and a light source. The scopes may also include a lens or other similar element (e.g., a lens cover) at or near its distal end.

One issue that can occur during visualization with a scope is condensation of moisture on the lens when the scope is introduced in the body of a patient or during the procedure when the user coagulates tissue. Condensation generally occurs due to the scope having an initial lower temperature than the body of the patient, causing moisture (so-called “fog”) to condense and collect on the scope when introduced into the body. The condensed fog can interfere with visualization, requiring the user to wait for the fog to clear from the visualizing element and/or wiping the fog off the scope. These issues can increase the time and difficulty of medical or surgical procedures using scopes.

To address the issue of fog forming during visualization procedures, various devices have been developed to heat a medical scope to temperatures at or above body temperature prior to insertion of the scope into the body of the patient. One exemplary device known from U.S. Pat. No. 8,152,717 to Gomez is a single-use, disposable device that includes a canal that receives a medical scope and has an associated heating element that heats the canal to heat an inserted medical scope prior to or during a visualization procedure; following the procedure, the entire device may be discarded to reduce the risk of cross-contamination between patients. One particular shortcoming associated with the device known from Gomez is the operating cost to the user due to the device being formed as an entirely disposable unit. Another exemplary device known from U.S. Patent Application Publication No. 2016/0135673 to Miller et al. includes multiple chambers that hold cleaning material and are heated by an electrical resistive heater segregated from the chambers. Some shortcomings associated with the device known from Miller et al. are the difficulty of sterilizing the device for re-use, if desired, or, alternatively, the replacement cost associated with disposing of the entire unit.

Another exemplary device is known from International Patent Application No. WO 2015/051098 to Temple et al. and includes a rechargeable heater with a disposable casing that fits into a cavity of the heater to receive and warm the medical scope. One particular shortcoming associated with the device known from Temple et al. is that the construction does not appear to account for instances where blood or other biological contaminants may inadvertently come into contact with the heater due to mishandling by the user or other operating room conditions, contaminating the relatively expensive heater in a manner that may not permit sterilization and re-use.

There exists a need in the art for a medical device warmer that can address at least some of the shortcomings of known warmers.

### SUMMARY

In some exemplary embodiments, there is provided an insert for warming a medical device, which includes an insert mating feature configured to releasably engage a corresponding mating feature of a housing.

In one exemplary embodiment, a disposable insert for warming a medical device includes: a base having a canister compartment formed therein; a canister held within the canister compartment, the canister having a chamber formed therein that defines a chamber opening; a resistive heater thermally bridged with the canister; a controller electrically coupled to the resistive heater and configured to provide electric current to the resistive heater; and an insert mating feature connected to the base and configured to releasably engage a corresponding mating feature of a housing, the insert mating feature including at least one beveled surface.

In another exemplary embodiment, a warmer for a medical device includes: a housing defining an insert compartment and a housing opening; a housing mating feature held within the insert compartment; and a warming insert placed within said insert compartment. The warming insert includes: a base having a canister compartment formed therein; a canister held within the canister compartment and having a chamber formed therein, the chamber defining a chamber opening aligned with the housing opening to form a device opening; a resistive heater thermally bridged with the canister; a controller electrically coupled to the resistive heater and configured to provide electric current to the resistive heater; and an insert mating feature connected to the base and releasably engaged to the housing mating feature.

In yet another exemplary embodiment, a kit for forming a warmer for a medical device includes: a housing defining an insert compartment and a housing opening; a housing mating feature held within the insert compartment; and at least one warming insert sized to fit within the insert compartment. The at least one warming insert includes: a base having a canister compartment formed therein; a canister held within the canister compartment and having a chamber formed therein, the chamber defining a chamber opening; a resistive heater thermally bridged with the canister; a controller electrically coupled to the resistive heater and configured to provide electric current to the resistive heater; and an insert mating feature connected to the base and configured to releasably engage the housing mating feature such that the chamber opening aligns with the housing opening to form a device opening.

One possible advantage that may be realized by some exemplary embodiments formed in accordance with the present invention is that the warming insert can be removed from the housing for disposal or separate sterilization.

Another possible advantage that may be realized by some exemplary embodiments formed in accordance with the present invention is that the warming insert can be formed as a relatively inexpensive disposable unit while the housing is re-sterilizable, allowing for the housing to be re-used multiple times and reduce the cost to medical care providers.

Yet another possible advantage that may be realized by some exemplary embodiments formed in accordance with the present invention is that the warming insert can be easily replaced within the housing by disengaging the insert mating feature from the housing mating feature.

### BRIEF DESCRIPTION OF THE DRAWINGS

FIG. 1 is a perspective view of an exemplary embodiment of a warmer for a scope device, formed in accordance with the present invention;

FIG. 2 is an exploded view of the warmer shown in FIG. 1;

FIG. 3 is a bottom view of an exemplary embodiment of a housing part of the warmer shown in FIGS. 1-2, formed in accordance with the present invention;

FIG. 4 is an exploded view of an exemplary embodiment of a warming insert of the warmer shown in FIGS. 1-2, formed in accordance with the present invention;

FIG. 5 is a perspective view of the warming insert shown in FIG. 4;

FIG. 6 is a side view of the warming insert shown in FIGS. 4-5;

FIG. 7 is a circuit diagram of an exemplary embodiment of a controller of the warmer shown in FIGS. 1-2, formed in accordance with the present invention;

FIG. 8 is a perspective view of a medical device inserted within and being warmed by the warmer shown in FIG. 1, in accordance with the present invention; and

FIG. 9 is a perspective view of an exemplary embodiment of a kit for forming a warmer including the housing shown in FIGS. 1-3 and multiple warming inserts similar to the warming insert shown in FIGS. 4-6, in accordance with the present invention.

### DETAILED DESCRIPTION OF EXEMPLARY EMBODIMENTS

Reference will now be made in detail to certain exemplary embodiments according to the present disclosure, certain examples of which are illustrated in the accompanying drawings. Wherever possible, the same reference numbers will be used throughout the drawings to refer to the same or like parts.

In this application, the use of the singular includes the plural unless specifically stated otherwise. In this application, the use of “or” means “and/or” unless stated otherwise. Furthermore, the use of the term “including,” as well as other forms such as “included” and “includes,” is not limiting. The section headings used herein are for organizational purposes only and are not to be construed as limiting the subject matter described. All documents, or portions of documents, cited in this application including but not limited to patents, patent applications, articles, books, and treatises are hereby expressly incorporated by reference in their entirety for any purpose.

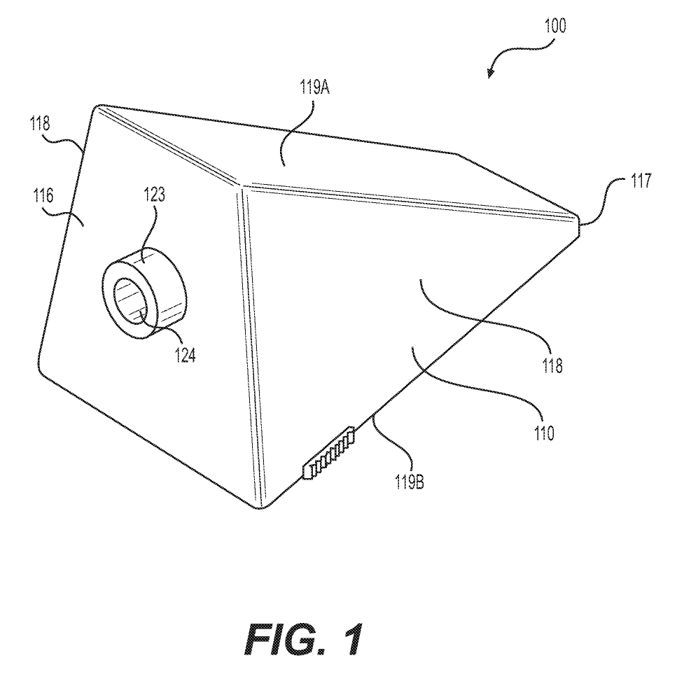

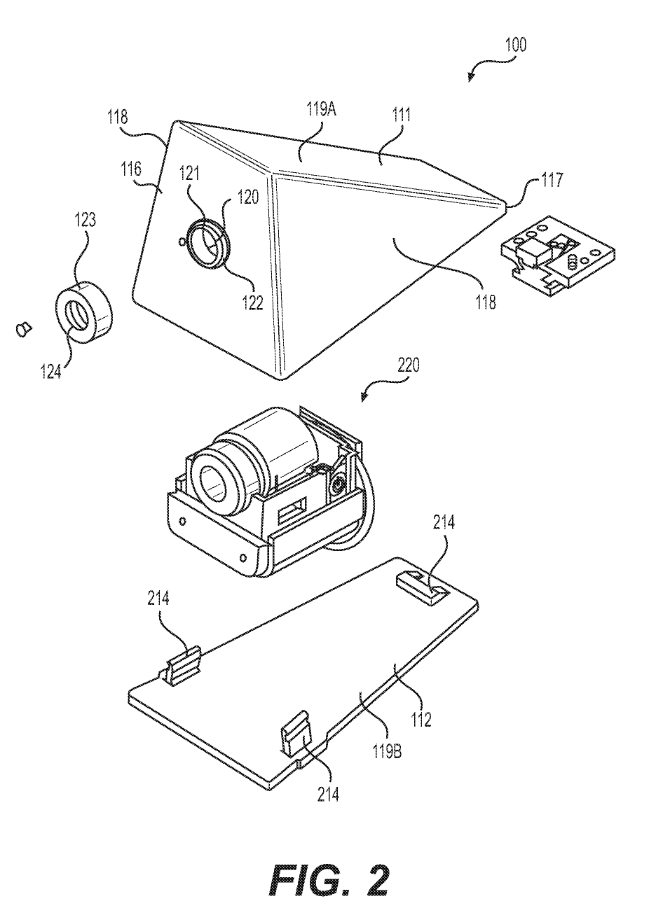

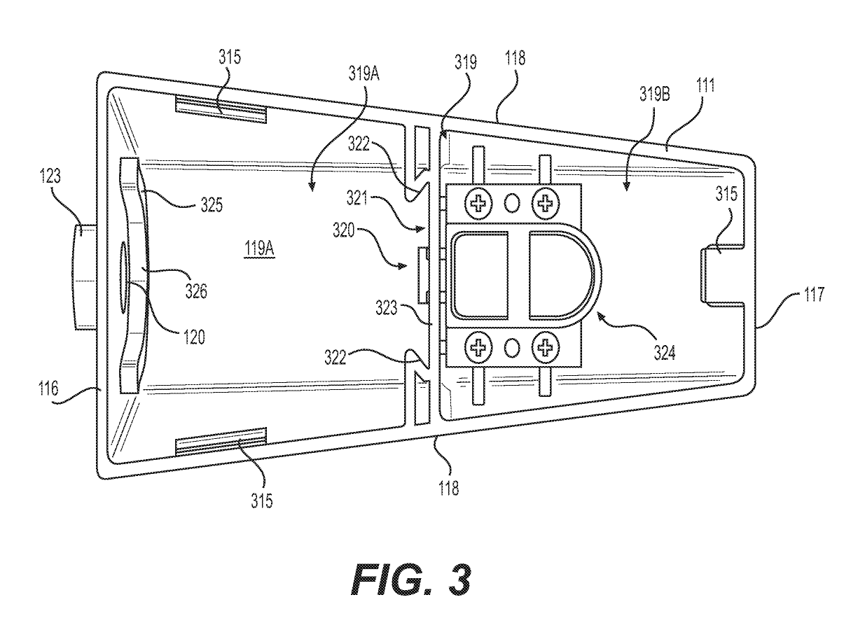

Referring now to the drawings, and more particularly to FIGS. 1-3, there is shown an exemplary embodiment of a warmer 100 formed in accordance with the present invention, which generally includes a housing 110 and a warming insert 220 placed within the housing 110. The housing 110 may be formed as a multiple-part housing with a first housing part 111 and a second housing part 112 reversibly connected to the first housing part 111 so the housing parts 111, 112 may be disconnected from each other to, for example, sterilize the housing 110 after use. As shown, the housing parts 111, 112 may be connected together by one or more second locking features 214 (shown in FIG. 2 as tabs) connected to the second housing part 112, which interlock with one or more first locking features 315 (shown in FIG. 3 as recesses and a tab) formed in the first housing part 111. When a user wishes to separate the housing parts 111, 112 from one another, the second locking feature(s) 214 can be unlocked from the first locking feature(s) 315 and allow separation of the housing parts 111, 112.

In some exemplary embodiments, the housing 110 can be formed to have the general shape of a triangular prism that decreases in height from a front wall 116 of the housing 110 to a rear wall 117 of the housing 110, with the front wall 116 being connected to the rear wall 117 by sidewalls 118, a top wall 119A, and a bottom wall 119B. While the housing 110 is shown as being in the general shape of a triangular prism, it should be appreciated that in some exemplary embodiments the housing 110 may be formed to have a different shape. In some exemplary embodiments, the second housing part 112 may define the bottom wall 119B of the housing 110. The housing 110 may include a housing opening 120 defined in the front wall 116. In some exemplary embodiments, the housing opening 120 may be formed with a lip 121 on an outer circumference 122 of the housing opening 120 onto which an opening cap 123 with a cap opening 124 can be fitted, reducing the risk of contaminants being trapped on the housing opening 120 following usage. The opening cap 123 may, for example, be a disposable cap comprising a relatively soft, inexpensive polymer, many of which are known; in one exemplary embodiment, the opening cap 123 comprises a silicone material. The opening cap 123 can protect a medical device during insertion into and removal from the warmer 100. The opening cap 123 may be removed after use and either disposed or sterilized and reused. In some embodiments, the opening cap 123 may have a diameter ranging from 3 mm to 30 mm. In further embodiments, the opening cap 123 may have a diameter ranging from 3 mm to 5 mm.

The housing 110, on the other hand, may comprise a re-sterilizable material that can be subjected to more than one sterilization operation and still exhibit acceptable characteristics for use in a surgical environment. Exemplary re-sterilizable materials may include, but are not limited to, thermoplastics such as polyetheretherketone and polyphenylsulfone. It should be appreciated that the re-sterilizable material of the housing 110 can be adjusted to withstand different sterilization operations, e.g., steam sterilization, EtO sterilization, irradiation, etc., and the housing 110 may comprise a variety of different materials in accordance with the present invention.

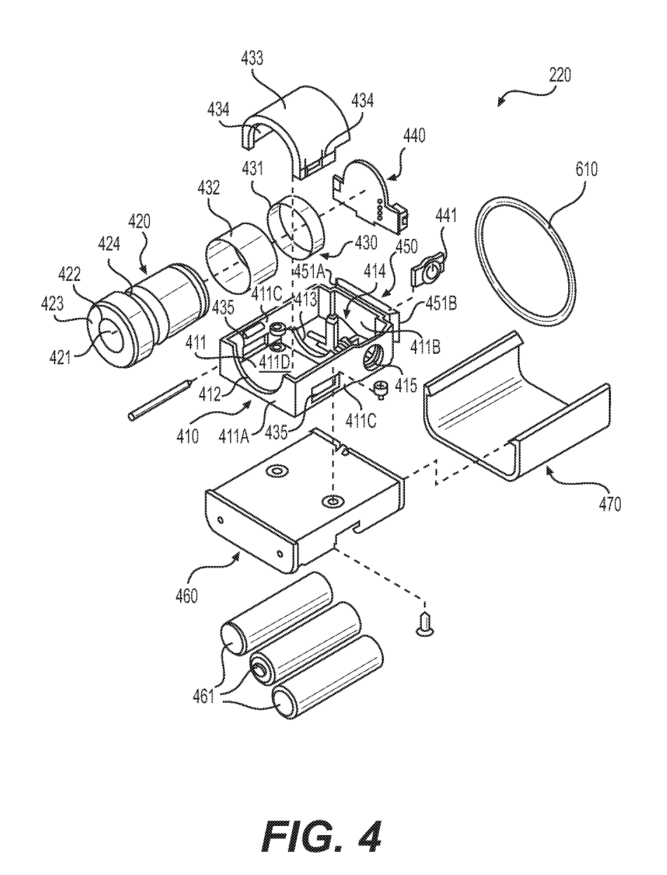

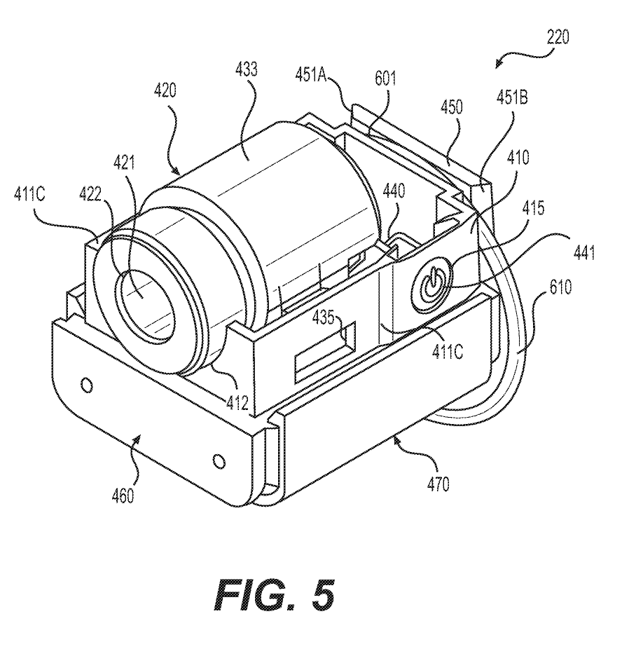

Referring specifically now to FIGS. 2 and 3, it can be seen that the housing 110 defines an insert compartment 319 (shown in FIG. 3) therein, which is sized and shaped to accept the warming insert 220. The insert compartment 319 may, in some exemplary embodiments, include a partition 320 connected to the sidewalls 118 and located between the front wall 116 and rear wall 117 to sub-divide the insert compartment 319 into a first sub-compartment 319A and a second sub-compartment 319B. In some exemplary embodiments, the partition 320 may extend only partially between the top wall 119A and bottom wall 119B and, in other exemplary embodiments, the partition 320 may fully extend from the top wall 119A to the bottom wall 119B. The partition 320 can have a housing mating feature 321 formed therein, which, in the illustrated exemplary embodiment, may be formed by one or more angled lateral portions 322 and a back portion 323, with the angled lateral portions 322 and back portion 323 together defining a recess forming the housing mating feature 321, the significance of which will be described further herein in regards to FIGS. 4-6. Optionally, a latch 324 may be held within the insert compartment 319 adjacent to the partition 320 and housing mating feature 321, for reasons that will be more fully described herein below. The housing 110 may also include a canister protrusion 325 defining a canister recess 326 therein adjacent to the housing opening 120 to support and align part of the warming insert 220. While not specifically shown, the insert compartment 319 of the housing 110 may be lined with or otherwise include insulating material, such as an insulating foam, to reduce the likelihood of the housing 110 becoming overly hot during operation of the warmer 100. The insulating foam also reduces heat loss to extend the warming life of the warmer 100.

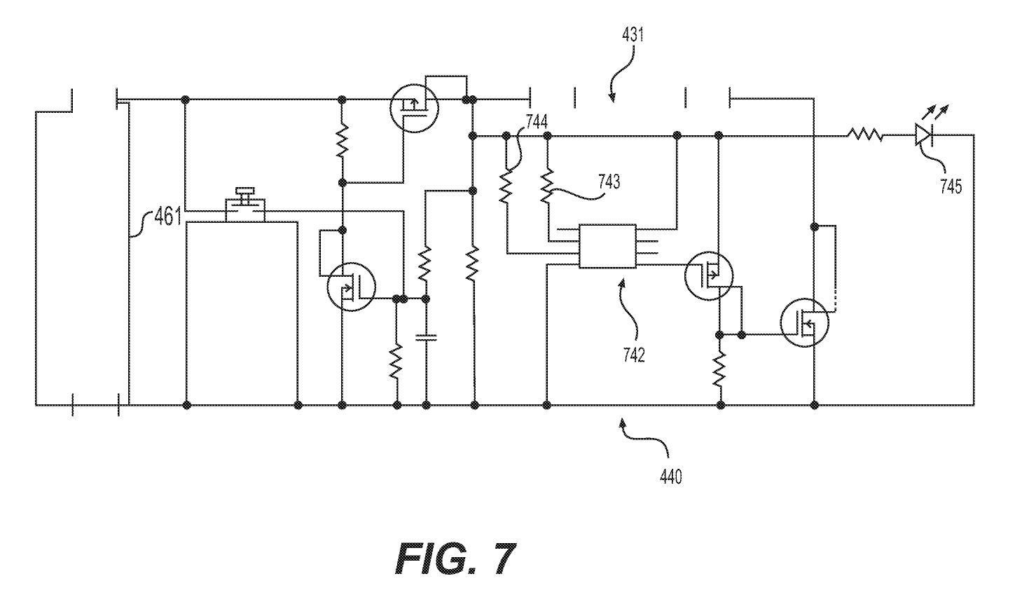

Referring now to FIGS. 4-6, an exemplary embodiment of a warming insert 220 formed in accordance with the present invention is shown and generally includes a base 410 having a canister compartment 411 formed therein, a canister 420 held within the canister compartment 411, a resistive heater 430 thermally bridged with the canister 420, a controller 440 electrically coupled to the resistive heater 430, and an insert mating feature 450 connected to the base 410. Optionally, the warming insert 220 may also include a battery compartment 460 connected to the base 410 for holding one or more batteries 461 and a tray 470 into which the battery compartment 460 is press fit. As shown, the base 410 may include a base front wall 411A connected to a base rear wall 411B by a pair of base sidewalls 411C and a base bottom surface 411D. The base front wall 411A may have an annular cutout 412 formed therein and the base bottom surface 411D can have an arc-shaped wall 413 upstanding therefrom, with the annular cutout 412 and arc-shaped wall 413 accommodating the canister 420, as can be seen in FIGS. 2 and 4. The base 410 can also have a controller compartment 414 formed therein, which can hold the controller 440, as shown in FIGS. 2 and 4. One of the base sidewalls 411C can include a button opening 415 to allow a button 441 of the controller 440 to be pressed when the controller 440 is held in the controller compartment 414, the significance of which will be described further herein in regards to FIG. 7. The base 410 may, in some exemplary embodiments, comprise a relatively inexpensive polymer material to lower manufacturing costs, as well as insulate heat that is produced by the warming insert 220 during operation. It should be appreciated that the illustrated general shape of the base 410 is exemplary only, and the base 410 can be formed to have other suitable shapes.

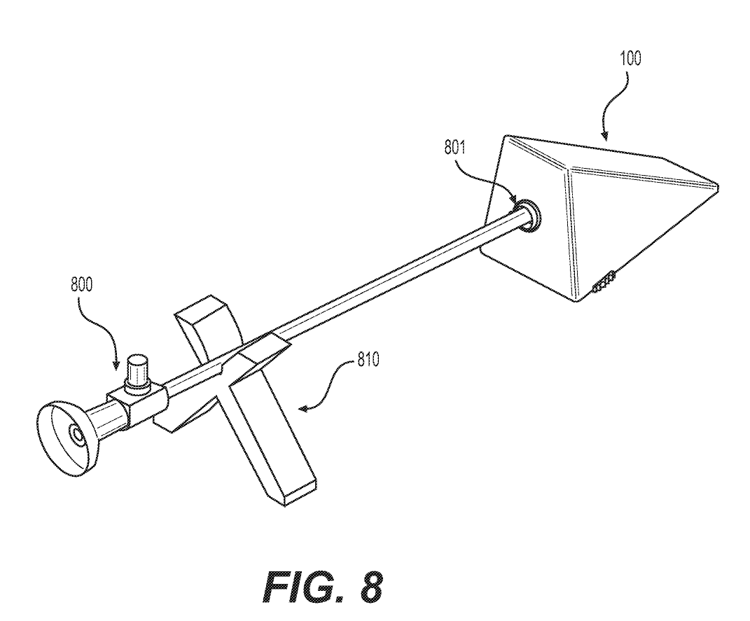

The canister 420, as shown, has a chamber 421 formed therein defining a chamber opening 422 adjacent a first end 423 of the canister 420. When the warmer 100 is assembled, as shown in FIG. 1, the chamber opening 422 of the canister 420 can partially or fully align with the housing opening 120 of the housing 110 to form a device opening 801 (shown in FIG. 8), which allows entry of a medical device into the chamber 421 of the canister 420. It should therefore be appreciated that the size of the chamber opening 422 (and the housing opening 120) can be adjusted to allow entry of various medical devices that may have differing sizes, as is known. The canister 420 may, in some exemplary embodiments, have a substantially cylindrical shape defining one or more radii and a recess 424 formed therein that accepts the material of the annular cutout 412 to stabilize the canister 420 within the canister compartment 411 of the base 410. An anti-fogging solution may be placed within the chamber 421 of the canister 420 to help prevent the formation of fog on a medical device that is inserted into the chamber 421. The anti-fogging solution may be, for example, a surfactant or any other substance that can reduce the formation of fog on a medical device, with many such substances being known. The anti-fogging solution may be directly placed in the chamber 421 or, alternatively, may be soaked or other otherwise immobilized in a solid material placed within the chamber 421. The canister 420 may comprise a relatively inexpensive polymer material, to reduce the cost of producing and replacing the canister 420.

The canister 420 can include a valve mechanism to prevent leaking of anti-fogging solution. For example, the canister may include a duckbill vale near the opening 422, which will allow insertion of an instrument but prevent leakage of contents from the chamber.

The resistive heater 430, as shown, is thermally bridged to the canister 420 and can include a resistive coil 431 formed of a high-resistance wire that can convert electric current received from the controller 440 into heat that will warm the canister 420. As used herein, the resistive heater 430 is “thermally bridged” to the canister 420 in the sense that the resistive heater 430 is directly or indirectly in material contact with the canister 420 so heat produced by the resistive heater 430 can efficiently heat the canister 420. In some exemplary embodiments, the resistive heater 430 can include a heat spreader 432, shown as a heat conductive ring, which fits on a portion of the canister 420 between the canister 420 and the resistive coil 431 and can spread heat produced by the resistive coil 431 across a greater surface area of the canister 420 to evenly distribute heat to the canister 420 and reduce the risk of “hot spots” forming on the canister 420 during warming. In some exemplary embodiments, the resistive heater 430 can be enclosed between the base 410 and a heater cover 433 that covers the resistive heater 430 and is locked to the base 410 by tabs 434 of the heater cover 433 fitting into cover openings 435 formed in the base 410. The heater cover 433 can comprise an insulating material, such as a polymer, to insulate the resistive heater 430, reducing heat loss to areas surrounding the resistive heater 430, while also reducing the risk of a user getting burned by touching the relatively hot resistive heater 430 during operation of the warmer 100.

The controller 440, which is shown as a printed circuit board (PCB), is electrically coupled to the resistive heater 430 and configured to provide electric current to the resistive heater 430. The electric current may originate from, for example, one or more batteries 461 electrically coupled to the controller 440 or any other suitable electric current source. To allow a user to control activation of the resistive heater 430, the previously described button 441 of the controller 440 can be linked to a switch of the controller 440 that allows current flow from the electric current source to the resistive heater 430 through the controller 440 when the button 441 is pressed. In some exemplary embodiments, and referring now to FIG. 7 as well, the controller 440 can be configured as a single-activation circuit that only allows the button 441 to turn on current flow from the electric current source to the resistive heater 430, i.e., the button 441 cannot be pressed again to stop current flow from the electric current source to the resistive heater 430. As shown in FIG. 7, the controller 440 may include a thermal switch 742 which, upon the resistive heater 430 reaching a certain temperature, can activate to restart or stop current flow from the electric current source to the resistive heater 430 and maintain the temperature of the resistive heater 430 at a desired level. The thermal switch 742 may connect to a lower limit resistor 743, the resistance value of which can be adjusted to control a lower temperature threshold at which current is re-supplied to the resistive heater 430, and an upper limit resistor 744, the resistance value of which can be adjusted to control an upper temperature threshold at which current flow to the resistive heater 430 is stopped. In some exemplary embodiments, the lower limit resistor 743 can be chosen to provide a lower temperature threshold slightly above body temperature, such as forty degrees Celsius, and the upper limit resistor 744 can be chosen to provide an upper temperature threshold higher than the lower temperature threshold, such as seventy degrees Celsius. The controller 440 may also include a light-emitting diode (LED) 745 that activates when current flows through the resistive heater 430, indicating that the resistive heater 430 is being supplied with electric current by the controller 440. In other respects, the controller 440 may be configured as desired.

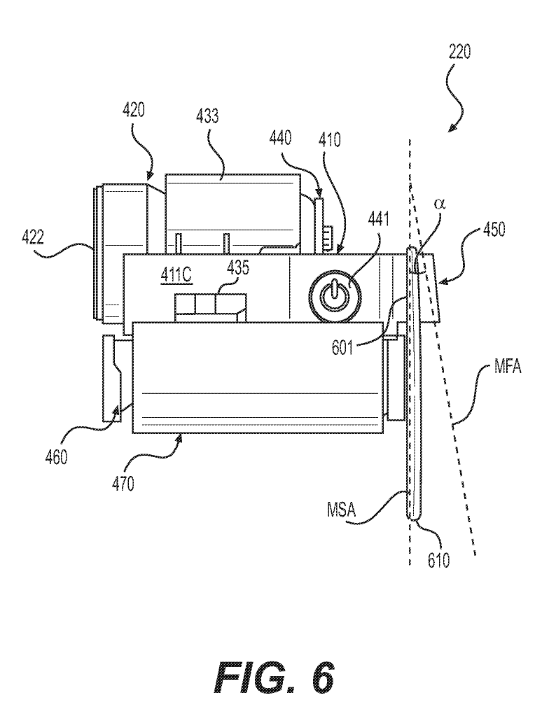

Referring specifically again to FIGS. 4-6, it can be seen that the insert mating feature 450 connects to the base 410 and is configured to releasably engage a corresponding mating feature of the housing 110, such as the previously described housing mating feature 321 formed in the partition 320. In some exemplary embodiments, a space may be formed between the insert mating feature 450 and the base 410. To allow releasable engagement with the housing mating feature 321, the insert mating feature 450 can have a generally rectangular shape including one or more beveled surfaces 451A, 451B, which are shown as a first lateral surface 451A and a second lateral surface 451B opposite the first lateral surface 451A. By forming the insert mating feature 450 with one or more beveled surfaces 451A, 451B, the insert mating feature 450 may slideably engage the housing mating feature 321 to hold the warming insert 220 within the insert compartment 319 such that the chamber opening 422 of the canister 420 aligns with the housing opening 120 to form a device opening that allows a medical device to be placed within the chamber 421 of the canister 420 and warmed while the resistive heater 430 is supplied with electric current from the controller 440. In this sense, the insert mating feature 450 is shaped to correspond to the shape of the housing mating feature 321 so the insert mating feature 450 can slide into the housing mating feature 321, or vice versa, to easily engage the mating features 321, 450 together and form the warmer 100 from the warming insert 220 and the housing 110. When the insert mating feature 450 slideably engages the housing mating feature 321, the insert mating feature 450 can be slideably disengaged from the housing mating feature 321 by sliding the insert mating feature 450 out of the housing mating feature 321, or vice versa. In some exemplary embodiments, the insert mating feature 450 may have one or more rounded surfaces, in addition to or instead of one or more beveled surfaces 451A, 451B. To prevent inadvertent disengagement of the mating features 321, 450, the previously described latch 324 can be placed and configured to allow the mating features 321, 450 to easily engage but, after engagement of the mating features 321, 450, spring out to prevent the insert mating feature 450 from slideably disengaging from the housing mating feature 321. The latch 324 may, for example, comprise a spring-loaded thumb latch that is depressed during insertion of the insert mating feature 450 into the housing mating feature 321 and, upon full engagement of the mating features 321, 450, springs out to prevent the insert mating feature 450 from sliding out of the housing mating feature 321 until depressed by, for example, a user wishing to remove the warming insert 220 from the warmer 100. It should therefore be appreciated that the insert mating feature 450 can allow engagement with and disengagement from the housing mating feature 321 in a relatively simple and quick fashion to quickly remove and replace the warming insert 220, as will be described further herein in regards to FIG. 6.

Referring specifically now to FIG. 6, it can be seen that the insert mating feature 450 can connect to a mating surface 601 of the base 410, which may be a part of the base rear wall 411B and defines a mating surface axis MSA therethrough. The insert mating feature 450 can define an insert mating feature axis MFA therethrough that forms an angle α relative to the mating surface axis MSA so the insert mating feature axis MFA is not parallel with the mating surface axis MSA; in some exemplary embodiments, the formed angle α may be an acute angle. By angling the insert mating feature 450 relative to the base rear wall 411B, the base 410 can be angled relative to the bottom wall 119B of the housing 110, which may rest on a surface such as an operating table during a visualization procedure, when the insert mating feature 450 engages the housing mating feature 321. Such angling of the base 410 can cause a corresponding angling of the held canister 420 relative to the horizontal, so any liquid held within the chamber 421 of the canister 420, such as the anti-fogging solution, is less likely to spill from the canister 420 during use of the warmer 100. Further, to assist with removal of the warming insert 220 from the housing 110, a pull tab 610, shown as a ring, can be placed in the space between the insert mating feature 450 and the mating surface 601 of the base 410 to provide a an easily grasped element for a user to pull up or down on the base 410 and connected insert mating feature 450 and disengage the insert mating feature 450 from the housing mating feature 321.

From the foregoing, it should be appreciated that the exemplary embodiment of the warmer 100 formed in accordance with the present invention includes a housing 110 and a warming insert 220, which may be conveniently removed from the housing 110 by disengaging an insert mating feature 450 of the warming insert 220 from a corresponding mating feature, such as housing mating feature 321, within the housing 110. In some exemplary embodiments, the components of the warming insert 220 can be of a relatively small size and comprise relatively inexpensive materials so the warming insert 220 can economically be produced as a single-use disposable unit that is replaced within the housing 110 between surgical operations, such as visualization using a medical scope. The housing 110, on the other hand, can comprise re-sterilizable materials able to withstand multiple sterilization operations, allowing re-use of the housing 110 in multiple surgical operations. In some embodiments, the warming insert 220 is sealed via heat shrink or other suitable sealing mechanism. The seal prevents a user from accessing the batteries 461 within battery component 460. The user is still able to access the button 441 to power the device.

Referring now to FIG. 8, the exemplary embodiment of a warmer 100 formed in accordance with the present invention is shown being used to warm a medical device 800, shown as a medical scope. To hold the medical device 800 stably within the warmer 100, a support stand 810 can be provided that supports the medical device 800 while being warmed by the warmer 100. Prior to inserting the medical device 800 within a device opening 801 formed by the aligned housing opening 120 and chamber opening 422, the switch of the controller 440 of the warming insert 220 can be activated so electric current is provided to the resistive heater 430 by the controller 440, warming the canister 420 and chamber 421 into which the medical device 800 will be inserted. The medical device 800 can be inserted into the chamber 421 through the device opening 801 while current is supplied to the resistive heater 430, warming the chamber 421 in the process, to warm the medical device 800. After a desired amount of time, the medical device 800 can be removed from the chamber 421 and used by a user, who may replace the medical device 800 into the chamber 421 during the procedure. Once the procedure is complete, the two housing parts 111, 112 of the housing 110 may be separated and the warming insert 220 may be removed from the insert compartment 319 by disengaging the insert mating feature 450 from the housing mating feature 321 and removing the warming insert 220 which, in some exemplary embodiments, may require a latch 324 to be depressed. In some exemplary embodiments, the housing 110 may be re-sterilized to be re-used with a new, sterilized warming insert, which may be structured identically to the previously removed warming insert 220.

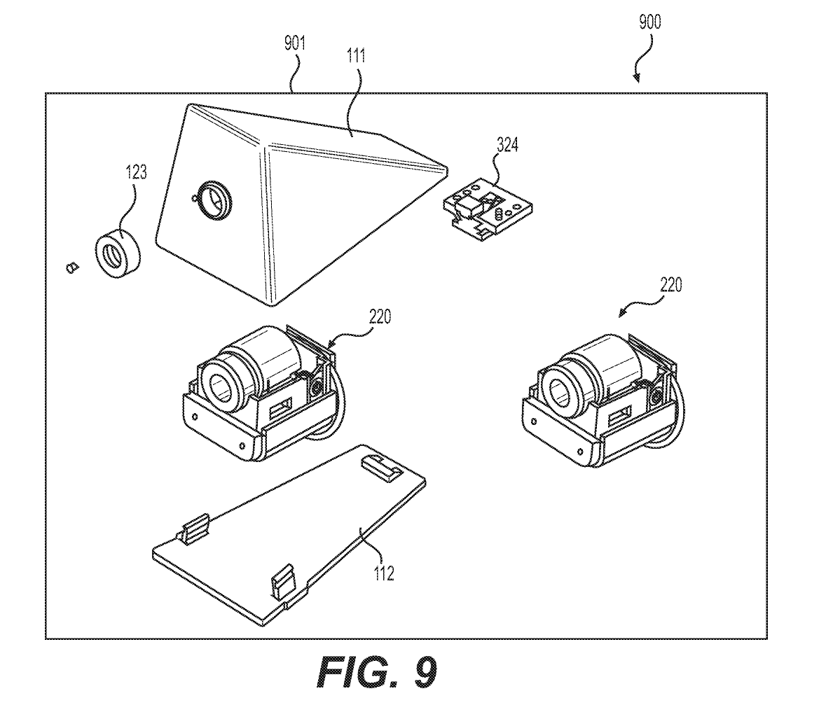

Referring now to FIG. 9, an exemplary embodiment of a kit 900 for forming a warmer 100 formed in accordance with the present invention is shown that generally includes the previously described housing 110 and one or more of the previously described warming inserts 220. The housing 110 and warming inserts 220 may, for example, be shipped or otherwise transported in a kit package 901 holding the components 110, 220 of the kit 900. Since the warming inserts 220 can be single-use, disposable units, having more than one similarly structured warming inserts 220 in the kit 900 can allow a user to perform a medical procedure using the housing 110 and one of the warming inserts 220 to form a warmer 100, as previously described, then remove the used, contaminated warming insert 220 from the housing 110 for disposal prior to the housing 110 being re-sterilized. Once the housing 110 is re-sterilized, another, sterile warming insert 220 can be placed in the insert compartment 319 of the housing 110 by engaging the insert mating feature 450 of the warming insert 220 with the housing mating feature 321 of the housing 110 such that the chamber opening 422 of the canister 420 of the warming insert 220 and the housing opening 120 of the housing 110 align to form a device opening 801 allowing a medical device 800 to be inserted in the chamber 421 of the canister 420 and be warmed. A user, such as a healthcare provider, can therefore keep multiple warming inserts 220 on hand to replace used, contaminated warming inserts in the housing 110 while keeping the re-usable housing 110 for additional uses following re-sterilization.

From the foregoing, it should be appreciated that an exemplary embodiment of a method for forming a warmer 100 may be provided in accordance with the present invention. A housing 110 defining an insert compartment 319 and a housing opening 120 has a housing mating feature 321 held in the insert compartment 319. A warming insert 220 is placed within the insert compartment 319, the warming insert 220 including a base 410 having a canister compartment 411 formed therein, a canister 420 held within the canister compartment 411 and having a chamber 421 formed therein that defines a chamber opening 422, a resistive heater 430 thermally bridged with the canister 420, a controller 440 electrically coupled to the resistive heater 430, and an insert mating feature 450 connected to the base 410. To form the warmer 100, the insert mating feature 450 of the warming insert 220 is engaged to the housing mating feature 321 held in the insert compartment 319 and the warming insert 220 is placed within the insert compartment 321 such that the chamber opening 422 aligns with the housing opening 120 to form a device opening 801. In some exemplary embodiments, the insert mating feature 450 may slideably engage the housing mating feature 321, as previously described. If the warming insert 220 is configured as a single-use, disposable unit, the method may further comprise removing the warming insert 220 from the insert compartment 319 after disengaging the insert mating feature 450 from the housing mating feature 321. Upon removing the warming insert 220, a replacement, sterile warming insert, which may be substantially identical the original warming insert 220, can be replaced in the insert compartment 319.

While the present invention has been described herein in conjunction with certain exemplary embodiments, a person of ordinary skill in the art can effect changes, substitutions or equivalents to the systems and methods described herein, which are intended to fall within the appended claims and any equivalents thereof.

1. A disposable insert for warming a medical device, comprising:
a base having a canister compartment formed therein;
a canister held within said canister compartment and having a chamber formed therein, said chamber defining a chamber opening;
a resistive heater thermally bridged with said canister;
a controller electrically coupled to said resistive heater and configured to provide electric current to said resistive heater; and
an insert mating feature connected to said base and configured to releasably engage a corresponding mating feature of a housing, said insert mating feature including at least one beveled surface.

2. The insert of claim 1, further comprising an anti-fogging solution placed within said chamber.

3. The insert of claim 1, wherein said controller comprises a printed circuit board (PCB) including at least one switch.

4. The insert of claim 3, wherein said PCB is configured as a single-activation circuit.

5. The insert of claim 1, wherein said insert mating feature includes a first lateral surface and a second lateral surface opposite said first lateral surface, at least one of said first lateral surface and said second lateral surface comprising said at least one beveled surface.

6. The insert of claim 1, further comprising a battery compartment coupled to said base.

7. The insert of claim 1, wherein said base includes a mating surface defining a mating surface axis and said insert mating feature is connected to said mating surface and defines an insert mating feature axis that forms an angle relative to said mating surface axis.

8. A warmer for a medical device, comprising:
a housing defining an insert compartment and a housing opening;
a housing mating feature held within said insert compartment; and
a warming insert placed within said insert compartment, said warming insert comprising:
a base having a canister compartment formed therein;
a canister held within said canister compartment and having a chamber formed therein, said chamber defining a chamber opening aligned with said housing opening to form a device opening;
a resistive heater thermally bridged with said canister;
a controller electrically coupled to said resistive heater and configured to provide electric current to said resistive heater; and
an insert mating feature connected to said base and releasably engaged to said housing mating feature.

9. The warmer of claim 8, wherein said insert mating feature is slideably engaged to said housing mating feature.

10. The warmer of claim 9, wherein said base includes a mating surface defining a mating surface axis and said insert mating feature is connected to said mating surface and defines an insert mating feature axis that forms an angle relative to said mating surface axis.

11. The warmer of claim 9, further comprising a latch held within said insert compartment and configured to prevent said insert mating feature from slideably disengaging from said housing mating feature.

12. The warmer of claim 8, wherein said housing comprises a first housing part connected to a second housing part, said first housing part and said second housing part together enclosing said insert compartment.

13. The warmer of claim 8, wherein said housing comprises a re-sterilizable material.

14. The warmer of claim 13, wherein said re-sterilizable material comprises at least one of polyetheretherketone and polyphenylsulfone.

15. The warmer of claim 8, further comprising at least one battery electrically coupled to said controller.

16. A kit for forming a warmer for a medical device, comprising:
a housing defining an insert compartment and a housing opening;
a housing mating feature held within said insert compartment; and
at least one warming insert sized to fit within said insert compartment, said at least one warming insert comprising:
a base having a canister compartment formed therein;
a canister held within said canister compartment and having a chamber formed therein, said chamber defining a chamber opening;
a resistive heater thermally bridged with said canister;
a controller electrically coupled to said resistive heater and configured to provide electric current to said resistive heater; and
an insert mating feature connected to said base and configured to releasably engage said housing mating feature such that said chamber opening aligns with said housing opening to form a device opening.

17. The kit of claim 16, wherein said insert mating feature is configured to slideably engage said housing mating feature.

18. The kit of claim 16, wherein said housing comprises a first housing part connected to a second housing part, said first housing part and said second housing part together enclosing said insert compartment.

19. The kit of claim 16, wherein said at least one warming insert comprises a plurality of warming inserts.

20. The kit of claim 16, wherein said base includes a mating surface defining a mating surface axis and said insert mating feature is connected to said mating surface and defines an insert mating feature axis that forms an angle relative to said mating surface axis.
